(19) United States
(12) Patent Application Publication
(10) Pub. No.: US 2025/0244262 A1
(43) Pub. Date: Jul. 31, 2025

(54) IN-SITU ANALYSIS OF DRILLING FLUID SOLIDS DURING WELLBORE DRILLING

(71) Applicant: Saudi Arabian Oil Company, Dhahran (SA)

(72) Inventors: Daniel Thomas Scott, Mandurah (AU); Tulio D. Olivares Antunez, Al Khobar (SA); Rafael M. Pino Rojas, Dhahran (SA); Fahad Aldossary, Al Khobar (SA)

(21) Appl. No.: 18/422,607

(22) Filed: Jan. 25, 2024

(51) Int. Cl. G01N 23/083; E21B 21/06; E21B 49/00; G01N 23/20; G01N 23/2055; G01N 33/28

(52) U.S. Cl. CPC G01N 23/083; E21B 21/065; E21B 49/005; G01N 23/20083; G01N 23/2055; G01N 33/2823

(57) ABSTRACT

Devices, systems and methods for in-situ analysis of drilling fluid solids is provided. A system includes a digital imaging system and a computer system operatively coupled to the digital imaging system. The digital imaging system is coupled to a shale shaker. The shale shaker is positioned at a surface of the Earth adjacent a wellbore and configured to receive a drilling fluid including solid objects. The digital imaging system is configured to capture images or measurement data of the solid objects. The computer system includes processors, and a computer-readable medium storing instructions executable by the processors to perform operations. The operations include receiving the images or measurement data captured by the digital imaging system and determining one or more properties of a target solid object of the solid objects. The one or more properties include specific gravity or composition of the target solid object.

### TECHNICAL FIELD

The present disclosure relates to wellbore drilling fluid analysis.

### BACKGROUND

Hydrocarbons trapped in subsurface reservoirs can be raised to the surface of the Earth (that is, produced) through wellbores formed from the surface to the subsurface reservoirs. Wellbore drilling systems are used to drill wellbores through a subterranean zone (for example, a formation, a portion of a formation or multiple formations) to the subsurface reservoir. At a high level, the wellbore drilling system includes a drill bit connected to an end of a drill string. The drill string is rotated and weight is applied on the drill bit to drill through the subterranean zone. Wellbore drilling fluid (also known as drilling mud) is flowed in a downhole direction through the drill string. The drilling fluid exits the drill bit through ports defined in the drill bit and flows in an uphole direction through an annulus defined by an outer surface of the drill string and an inner wall of the wellbore. As the drilling fluid flows towards the surface, it carries any cuttings and debris released into the wellbore due to and during the drilling. The cuttings and debris are released from the subterranean zone as the drill bit breaks the rock while penetrating the subterranean zone. When mixed with the drilling fluid, the cuttings and debris form a solid slurry that flows to the surface. At the surface, the cuttings and debris are filtered and the wellbore drilling fluid can be recirculated into the wellbore to continue drilling. The cuttings and debris carried to the surface by the drilling fluid provide useful information, among other things, about the wellbore being formed and the drilling process.

### SUMMARY

The present disclosure describes methods, devices, systems and techniques for in-situ analysis of solid objects in drilling fluids during wellbore drilling.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a schematic diagram of an example of a system for in-situ solid objects analysis in drilling fluids.

FIGS. 2A and 2B are schematic diagrams of an example solid object monitoring system that is coupled to a shale shaker.

FIG. 3A is a schematic diagram of an example shale shaker carrying different types of solid objects.

FIG. 3B is a schematic diagram of an example digital imaging system capturing solids objects in a shale shaker.

FIG. 4 is a flow chart of an example process for analysis of solid objects.

FIG. 5 illustrates a schematic diagram of an example computer system.

Like reference numbers and designations in the various drawings indicate like elements. It is also to be understood that the various exemplary implementations shown in the figures are merely illustrative representations and are not necessarily drawn to scale.

### DETAILED DESCRIPTION

During the construction process, solids in drilling fluids from surrounding formations provide physical data of the subsurface of a well. Thus, analysis of the solids offers multiple insights into the well's overall health. One example application of solid analysis is the assessment of well cleanliness, indicating whether the drilling hole is adequately cleaned and/or if cuttings have been successfully brought to the surface. This analysis can be carried out by experienced mud engineers who manually inspect solids in drilling fluids that pass over shale shakers by observing the number, size, or shape of solids in the drilling fluids. Based on these characteristics, mud engineers assess hole cleaning efficiency and make decisions regarding the need for additional additives in the drilling fluid to improve wellbore stability.

This disclosure describes using a system for in-situ analysis of solids in drilling fluids. The system can be implemented to analyze specific gravity and compositions of the solids in the drilling fluid. In addition, the size, shape, and/or quantity of solids can be analyzed. In some aspects, the system includes a digital imaging system and a computer system operatively coupled to the digital imaging system. The digital imaging system is positioned adjacent a shale shaker. The shale shaker is positioned at a surface of the Earth adjacent a wellbore and configured to receive a drilling fluid comprising a mixture of wellbore drilling mud and solid objects found in the wellbore while drilling the wellbore through a subterranean zone. The solid objects include drill solids and non-drilled solids, where the drill solids include drill cuttings and additives. The digital imaging system is configured to capture images or measurement data of the solid objects when the drilling fluid is received by the shale shaker. The computer system includes one or more processors and computer-readable medium storing instructions executable by the processors to perform operations. The operations can include (1) receiving the images or measurement data captured by the digital imaging system and (2) determining one or more properties of solid objects. The properties include specific gravity and/or compositions. In addition, the properties can include size, shape and/or quantity.

Implementations of the present disclosure can provide one or more of the following technical advantages and/or benefits. For example, the techniques described here deploys an in-situ analysis system which uses a digital imaging system to automatically measure properties of solid objects passing over the shale shaker. This improves efficiency in the analysis process and reduces human error. In addition, this system can be configured to determine if particular loss circulation materials (LCM) are stripped from the drilling fluid unexpectedly. This assists in assessing whether a concentration adjustment is required for a particular LCM or if the addition of a new LCM to the drilling fluids is required.

The analysis of LCMs can be achieved by measuring the specific gravity and/or compositions of the solids in drilling fluids. This system can also be configured to analyze ultra-fine solids (e.g., 1-2 micros) which would otherwise pose challenges for manual observations by mud engineers. Further, by providing real-time estimates of solids attributes such as specific gravity, quantity, size, shape, density, mineralogical, chemical and physical composition, the system facilitates identifying real-time issues with hole cleaning and enables mud engineers to act proactively to reduce stuck pipe incidents. It also reduces Non-Productive Time (NPT) and thus saves manufacturing costs.

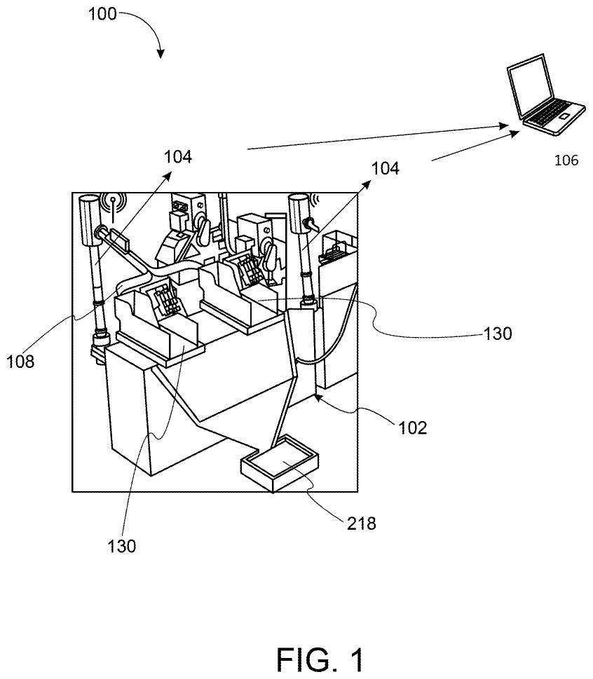

FIG. 1 illustrates schematic diagram of an example of a system for in-situ solid objects analysis in drilling fluids. As shown, the system 100 includes a digital imaging system 104 and a computer system 106 which is operationally coupled to the digital imaging system 104, for example, by wired or wireless operative coupling techniques. The digital imaging system 104 is positioned adjacent a shale shaker 102. The shale shaker 102 can be positioned at a surface of the Earth adjacent a wellbore. The digital imaging system 104 is configured to capture images or measurement data of the solid objects when the drilling fluid is received by the shale shaker 102.

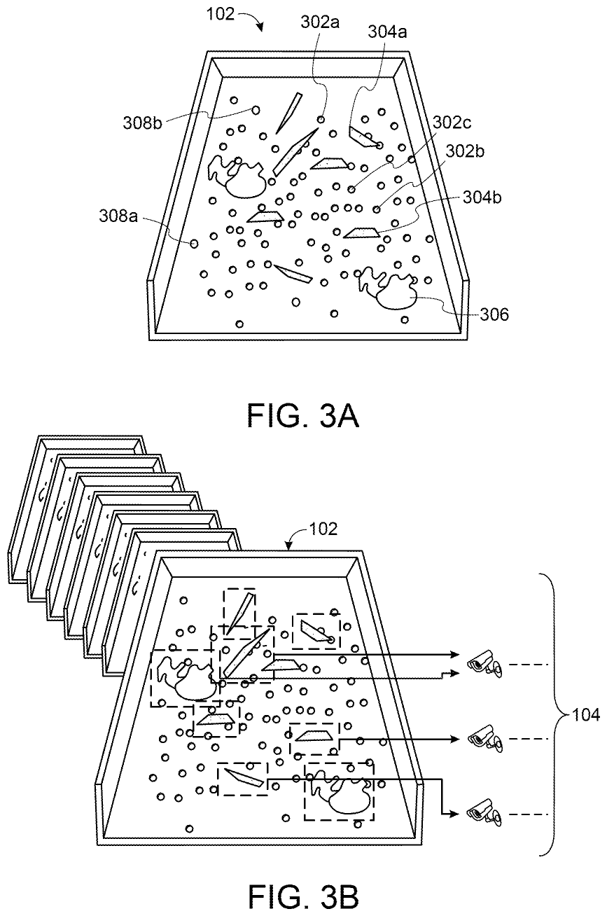

The shale shaker 102 can include an inlet 108 which introduces a drilling fluid (not shown) from a wellbore into the shale shaker 102. The drilling fluid can include multiple solid objects. As discussed in FIG. 3A below, the solid objects can include drill solids and non-drilled solids, where the drill solids include drill cuttings and additives. The drill cuttings are the fragmented rock and debris that are generated during the drilling process. The additives are solids added to the drilling fluids for various purposes. For example, the additives include LCM which is used to help control or prevent loss of fluid into subsurface formations. LCMs can come in various forms, such as fibers, granules, or flakes, and they work by plugging or sealing off the pathways through which drilling fluid is escaping. The non-drilled solids are other solids in the drilling fluid that are not additives or the drill cuttings. For example, the non-drilled solids include cavings or cement.

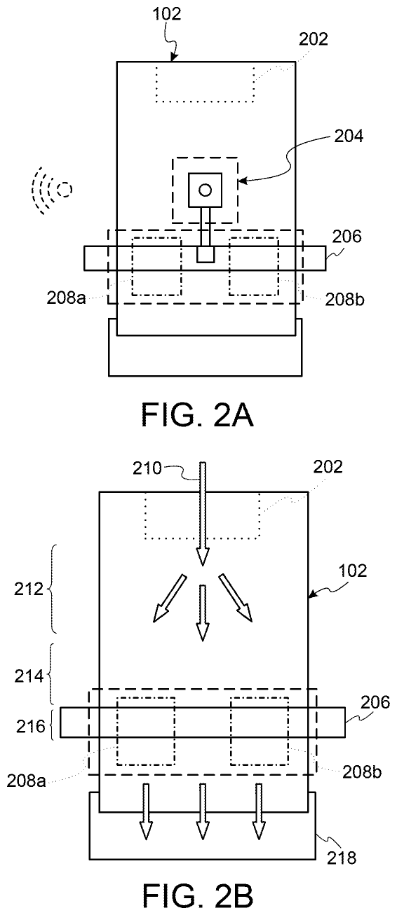

The shale shaker 102 can include one or more shakers 130 (two are shown in FIG. 1A). The drilling fluid flows from the inlet 108 into the shakers 130. As described in FIGS. 2A-2B below, the solid objects from which the drilling mud has been separated are discarded in the solids discard zone 218 downstream of the shale shaker screen. The drilling mud (and any fine solids, depending on the mesh size of the shale shaker screen) are gathered into a sump tank (not shown) for further treatment and recycling for reuse in the wellbore drilling operation.

The digital imaging system 104 can be amounted on the shale shaker 102 or positioned adjacent to the shale shaker 102 (as shown). The digital imaging system can include a camera which is configured to capture images of solid objects. The captured images are transmitted to the computer system 102 for size, shape, and quantity analysis of the solid objects. In some implementations, the digital imaging system 104 includes an X-ray system, which is configured to determine the specific gravity of the target solid objects. In some implementations, the digital imaging system 104 includes an X-ray diffraction system, which is configured to determine the mineralogical composition of the solid objects based on X-ray diffraction patterns. In some implementations, the digital imaging system 104 includes a hyperspectral imaging system, which is configured to determine the chemical and/or physical composition of the target solid objects using hyperspectral images.

The computer system 106 receives images or measurement data captured by the digital imaging system 104 and determines properties of solid objects. The properties include specific gravity and/or compositions. The properties can further include quantity, size or shape of the solid objects. In some implementations, the computer system 106 stores pre-determined properties of the solid objects in a computer-readable database or a library. For example, the database can include properties such as specific gravity, compositions, XRD patterns or hyperspectral data, etc. The computer system 104 compares one or more properties of the solid objects with the pre-determined properties in the database or the library, as described below. In some implementations, in response to a comparison result, the computer system 106 raises an alert to an operator that recommends a modification of a drilling parameter. The drilling parameter can be a concentration of an LCM. For example, the computer system 106 can determine if particular LCM are stripped from the drilling fluid unexpectedly by determining the specific gravity and/or compositions of solid objects. If all anticipated LCMs are detected over the shale shakers, the computer system 106 can associate this detection with an increased likelihood that current LCM selection is appropriate and provides a recommendation to operators that it is unnecessary to add these LCMs in the subsequent well operations. On the other hand, if the absence of a particular LCM is detected, the computer system 106 can associate this detection with an increased likelihood that this particular LCM has obstructed the formations and raise an alert to operators recommending a modification in the selection of LCMs or an increased concentration of a specific LCM in the upcoming well operations.

In some wellbore drilling assemblies, multiple shale shakers 102 can be implemented. In such instances, a single digital imaging system 104 coupled to one of the shale shakers 102 can be used for image and/or measurement data capture. The computer system 106 can classify solid objects based on images and/or measurement data captured by the single digital imaging system 104 and extrapolate or calibrate the classification to the solid objects carried by other shale shakers 102. In some implementations, a respective digital imaging system 104 can be mounted to each shale shaker 102, and all the shale shakers 102 can transmit captured images to the computer system 106. In such implementations, the computer system 106 can develop a classification of solid objects carried by each individual shale shaker 102 and also develop a drilling assembly-wide classification of solid objects carried by all the shale shakers 102.

FIGS. 2A and 2B are schematic diagrams of a shale shaker with an example digital imaging system. FIG. 2A shows that the shale shaker 102 includes a header box/possum belly 202 through which a drilling fluid that includes a mixture of the solid objects and the wellbore drilling mud enter the shale shaker 102. Specifically, the drilling fluid lands on a shaking screen and is carried downstream of the shale shaker 102 by a vibration of the shaking screen operated by shaker basket motors 208a, 208b. The shale shaker 102 includes a static motor cable support member 206 (for example, a swing arm or other static, non-vibrating member) which spans a width of the shaking screen and that carries cabling or wiring to power the motors 208a, 208b. In some implementations, as shown in FIG. 2A and FIG. 2B, the digital imaging system 104 is mounted on and directly attached to the support member 206. In some implementations, as shown in FIG. 1, digital imaging system 104 are separately positioned adjacent to the shale shaker 102. In some implementations, an image capture zone 204 is defined by the support member 206.

The digital imaging system 104 can include a smart, waterproof, high resolution, wireless camera or any other image or vision sensor such as infrared sensor, gamma ray sensor, computerized tomography (CT) scanner, X-ray system, XRD system, or a hyperspectral imaging system, to name a few. The digital imaging system 104 is oriented such that its view finder or cameras faces the drilling fluid. In particular, the view finder or cameras is capable of capturing a plan view of the shaking screen and of the solid objects moved by the shaking screen. The digital imaging system 104 can have a field of view that spans an entire width of the shaking screen so as to image an entirety of the drilling fluid carried by the shaking screen. The field of view can also span a length segment of the shaking screen on which multiple solid objects are carried.

FIG. 2B shows different length segments of the moving tray or the mesh or sieve of the shale shaker 102. In particular, the length segment nearest the header box/possum belly 202 can be a very wet or splash zone 212 in which the drilling fluid is the most wet, that is, has the largest concentration of drilling mud among all the length segments. The arrow 210 represents a direction of movement of the drilling fluid as the shaking screen vibrates. The length segment downstream of the very wet or splash zone 212 is an intermediate zone 214 that is drier compared to the very wet or splash zone 212 because at least some but not all of the drilling mud has been drained from the drilling fluid. The length segment downstream of the intermediate zone 214 is the dry zone in which the drilling fluid is most dry, that is, has the least concentration of drilling mud among all the length segments. The dry zone 214 can be the length segment that is immediately upstream of the end of the shaking screen. Most, if not all, of the drilling mud liquid has been drained from the slurry leaving only solid objects or mostly solid objects with very little drilling mud in the dry zone 214. The solid objects from which the drilling mud has been separated are discarded in the solids discard zone 218 downstream of the shale shaker screen. The drilling mud (and any fine solids, depending on the mesh size of the shale shaker screen) are gathered into a sump tank for further treatment and recycling for reuse in the wellbore drilling operation.

FIG. 3A is a schematic diagram of an example shale shaker carrying different types of solid objects. FIG. 3B is a schematic diagram of an example digital imaging system capturing solids objects in a shale shaker. As shown in FIG. 3A, the shale shaker 102 is carrying solid objects of different types in the drilling fluids including drill cuttings (302a, 302b, 302c), non-drilled solids (304a, 304b), and additives (308a, 308b). The drilling fluids can also carry non-solid objects (306), for example, liquid hydrocarbon from the wellbore. The drill cuttings are the fragmented rock and debris that are generated during the drilling process. The additives are solids added to the drilling fluids for various purposes. For example, the additives include LCM which is used to help control or prevent loss of fluid into subsurface formations. LCMs can come in various forms, such as fibers, granules, or flakes, and they work by plugging or sealing off the pathways through which drilling fluids is escaping. The non-drilled solids are other solids in the drilling fluid that are not additives or the drill cuttings. It is understood that FIG. 3A is for illustrative purposes only and doesn't accurately represent the true relative size, shape or quantity ratio of various types of objects in drilling fluids.

As shown in FIG. 3B, the digital imaging system 104 captures multiple images or measurement data of solid objects in the shale shaker 102 over a period of time and transmits the images or the measurement data to the computer system 106 in real-time. For the purposes of this disclosure, the terms “real-time,” “real time,” “realtime,” “real (fast) time (RFT),” “near(ly) real-time (NRT),” “quasi real-time,” or similar terms (as understood by one of ordinary skill in the art) mean that an action and a response are temporally proximate such that an individual perceives the action and the response occurring substantially simultaneously. For example, the time difference for a response to display (or for an initiation of a display) of data following the individual's action to access the data may be less than 1 ms, less than 1 sec., less than 5 secs., etc. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit (or a combination of these or other functions) the data,

As discussed, the digital imaging system 104 can includes at least one of an X-ray system, an X-ray diffraction (XRD) system, a hyperspectral imaging system, a camera, or an image sensor. The camera or image sensors capture images of solid objects in the shale shaker 102. The X-ray system captures measurement data (e.g., light intensity of the transmitted X-Ray) and/or X-ray images of solid objects. The XRD system captures measurement data (e.g., light intensity of diffracted X-rays as a function of the angle of diffraction) and/or XRD images of solid objects. The hyperspectral imaging system captures measurement data (e.g., light intensity of the dispersed lights) and/or a series of hyperspectral images of solid objects, each image representing the solid object at a specific wavelength or within a specific spectral range.

In some implementations, the camera and/or the hyperspectral imaging system are capable of capturing images containing multiple solid objects within a single frame. In contrast, the X-ray system and/or XRD system acquires measurement data or captures image focusing on a single target solid object. The target solid object can be a random solid object, either a drill solid or a non-drilled solid, in the drilling fluids. In some implementations, the system classifies the solids objects into drill solids and non-dilled solids using the smart camera, as discussed below. In an example, the target solid object can be a random drill solid. In another example, the target solid object can be a drill solid with particular characteristics (such as size or shape) selected for more in-depth analysis. In some implementations, the X-ray system and/or XRD system are configured to sample solid objects for analysis over a period of time, rather than measuring each individual solid object.

As noted above, the digital imaging system 104 can include an X-ray system. The system including the X-ray system is configured to determine the specific gravity of the solid objects. In some implementations, the X-ray system includes an X-ray tube and a detector. The X-ray tube can be configured to generate a homogenous X-ray beam (also called monochromatic beam) and/or a heterogenous X-ray beam. A monochromatic beam refers to a beam of X-ray that consists of a single, specific wavelength or frequency. In contrast, a heterogenous beam has different wavelengths or frequencies.

For a monochromatic X-ray beam configuration, an incident monochromatic X-ray is generated with an incident light intensity I₀ at an energy level E₁ that passes through the solid objects in the drilling fluid as they move across the shale shaker. The detector is configured to receive a transmitted X-ray that have passed through the solid objects and measure a transmitted light intensity I of the transmitted X-Ray. In some implementations, the incident light intensity I₀ and the energy level E₁ are predetermined values which are stored in the computer system 106 and/or the digital imaging system 104. The digital imaging system 104 transmits the measured transmitted light intensity I to the computer system 106 for data processing.

In some implementations, the system 100 further comprises a non-contact thickness gauge configured to measure the thickness t of the target solid object. For example, the thickness gauge is based on ultrasonic technology, where ultrasonic waves are emitted towards the solid objects, and the time taken for the signal to return is used to calculate thickness. In another example, the thickness gauge involves laser-based technology, where a laser beam is directed at the material, and the reflected beam is analyzed to determine thickness. Other examples of thickness gauge includes: eddy current thickness gauges, which uses electromagnetic induction to generate eddy currents in the material and the interaction between these currents and the material's properties is used to determine thickness; infrared thickness gauges which measures the thermal conductivity of a material and the rate at which heat is conducted through the material is correlated with its thickness.

In some implementations, the computer system 106 is configured to determine the specific gravity of a target solid object using the equations:

$${μ}_{1} = - \frac{1}{t} ln ( \frac{I}{{I}_{0}} ) \qquad ( 1 )$$

$$ρ = \frac{{μ}_{1}}{{μ}_{water}} \qquad ( 2 )$$

wherein p is the specific gravity of the target solid object, I₀ is the incident light intensity, I is the transmitted light intensity, t is a thickness of the target solid object, u water is linear attenuation coefficient of water, μ₁ is linear attenuation coefficient of the target solid object at the energy level E₁.

The equation (1) described above is based on Beer-Lambert's law relating the absorption of light to the material through which the X-ray beam passes. Under Beer-Lambert's law, there is an exponential attenuation of intensity for x-ray photons as they traverse a target solid object as shown in equation (1). In addition, μ/ρ is a constant, which is known as the mass attenuation coefficient and has units of cm2/g. Thus, the mass attenuation coefficient is independent of specific gravity for a particular element or compound while linear attenuation coefficient increases with increasing specific gravity. The values of μ/p, for photon energies 1 keV to 20 MeV, are documented in a database by National Institute of Standards & Technology for all elements Z=1 to 92 and for 48 compounds and mixtures of radiological interest. As described earlier, the characteristic property of x-ray attenuation coefficients μ₁ can be calculated to determine specific gravity of solid objects.

In some implementations, the X-ray tube of the X-ray system is configured to generate a heterogeneous beam. A heterogeneous X-ray beam refers to an X-ray beam that consists of photons with a range of energies. In some implementations, the heterogeneous beam generated by the X-ray tube includes the first incident X-ray at the first energy level E₁ and a second incident X-ray at a second energy level E₂ that pass through the target solid object. The first energy level E₁ and the second energy level E₂ can be predetermine values that are stored in the computer system 106 and/or the digital imaging system 104. The heterogeneous incident X-ray passes through the solid objects in the drilling fluid as they move across the shale shaker. A detector is configured to receive a transmitted X-ray that have passed through the solid objects and measure a transmitted light intensity I of the transmitted X-Ray. The digital imaging system 104 transmits the measured transmitted light intensity I by the detector to the computer system 106 for data processing.

In some implementations, for a heterogeneous beam configuration, the computer system 106 determines the specific gravity of the target solid object using the equations:

$$ρ = \frac{( {μ}_{1} - c {μ}_{2} )}{β ( 1 - c )} \qquad ( 3 )$$

$$where c = {( \frac{{E}_{2}}{{E}_{1}} )}^{3.1}$$

wherein ρ is the specific gravity of the target solid object, μ₁ is the linear attenuation coefficient of the target solid object at the first energy level E₁. μ₂ is linear attenuation coefficient of the target solid object at the second energy level E₂, and β is scattering attenuation constant. μ₁ and μ₁ can be obtained using the questions (1) and (2) as discussed above by measuring transmitted light intensity at two energy levels E₁ and E₂.

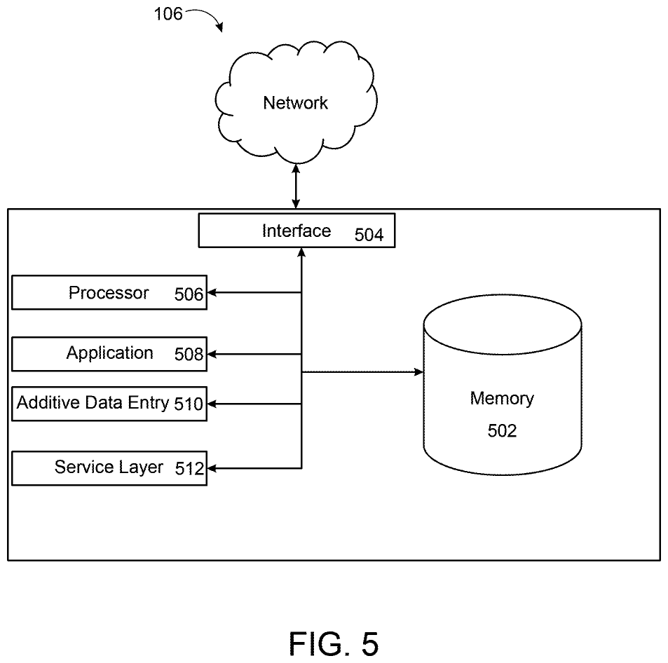

There are typically diverse additives mixed into drilling fluids and each has its unique specific gravity. Likewise, there are also distinct specific gravity associated with drilling cuttings and non-drill solids. In some implementations, the digital imaging system 104 coupled with X-Ray systems is deployed to estimate the specific gravity of all types of solids once the drilling fluid returns from the borehole. The computer system 106 calculates the specific gravity of solids based on measured data from the digital imaging system 104 and stored data (e.g., the incident light intensity I₀, linear attenuation coefficient of water μ_{water}). The computer system 106 further performs real-time comparison between the pre-determined specific gravity and the calculated specific gravity to determine the materials of the solids. In some implementations, the pre-determined specific gravity is stored in a non-transitory medium (such as a non-volatile memory 502 as described in FIG. 5 below) of the computer system 106 for different type of solid objects. In some implementations, the system can classify the solids into three groups: additives, drilling cuttings and non-drilled solids based on calculated specific gravity.

In one implementation, the comparison process can be performed by using a table, a database, or another suitable data structure. In the implementation using a table, it can have, for example, a plurality of rows and columns where each row has at least a first column to represent a pre-determined specific gravity and a corresponding second column to represent an associated chemical or compounds for the solids. During comparison, the computer system 106 can use the calculated specific gravity and compare it to each row in the column of the pre-determined specific gravity until it matches. From there, the chemical or compounds for the solids can be determined by accessing an associated column of the selected row.

The system 100 with an X-ray system can detect the presence or absence of a particular solid (such as LCMs) based on specific gravity. Determining whether specific LCMs are unnecessarily extracted from the drilling fluid assists in reconsideration of the LCMs chosen for future drilling fluids. For instance, if all anticipated LCMs are detected over the shale shakers, the computer system 106 can associate this detection with an increased likelihood that current LCM selection is appropriate and provides a recommendation to operators that it is unnecessary to add these LCMs in the subsequent well operations. On the other hand, if the absence of a particular LCM is detected, the computer system 106 can associate this detection with an increased likelihood that this particular LCM has obstructed the formations and raise an alert to operators indicating a potential need for adjusting the selection of LCMs or a concentration of an LCM in the subsequent well operation. This in-situ analysis of specific gravity of solids to determine the presence of specific LCMs improves operational efficiency by optimizing the utilization of materials (such as LCMs) in the drilling fluids.

In some implementations, the digital imaging system 104 includes an X-ray Diffraction (XRD) system. The system 100 including the XRD system can be configured to determine the mineralogical composition of the solid objects.

In one implementation, the XRD system includes an X-ray tube and a detector. The X-ray tube is configured to emit a plurality of incident X-rays with a specific wavelength (e.g., a monochromatic X-ray beam) that interacts with the solids objects at a plurality of incident angles. Each incident X-ray corresponds to a respective incident angle θ. In some implementations, the X-rays produced have energies on the order of kiloelectronvolts (keV). The monochromatic X-ray beam is directed onto the solid object of interest. The incident X-rays interact with the electrons in the atoms of the solid object. When X-rays encounter the crystal lattice of a crystalline material, they undergo elastic scattering. This scattering is a result of the interaction between the incident X-rays and the electrons in the crystal lattice. According to Bragg's Law, the scattered X-rays constructively interfere if the following condition is met:

$$2 d sin θ = n λ \qquad ( 4 )$$

where d is the spacing between diffracting planes, θ is the incident angle, n is an integer, and λ is the beam wavelength. X-rays are typically used to produce the diffraction pattern because their wavelength, λ, is often the same order of magnitude as the spacing, d, between the crystal planes (1-100 angstroms).

The detector is configured to receive a plurality of diffracted X-rays that have interacted with the solid objects (e.g., a target solid object) at the plurality of incident angles θ and measure a diffracted light intensity for each of the plurality of diffracted X-rays. In other words, the detector records the intensity of diffracted X-rays as a function of the angle θ of diffraction.

In some implementations, the computer system 106 receives the intensity of diffracted X-rays and their corresponding angles and then generate an X-ray diffraction pattern based on these data. The resulting diffraction pattern is a plot of intensity versus diffraction angles. Peaks in the XRD pattern correspond to constructive interference between X-rays scattered by the crystal lattice. The positions and intensities of the diffraction peaks are thus indicative of the lattice spacing and crystal symmetry. In some implementations, the XRD patterns are generated by the X-ray diffraction system which then transmits such patterns to the computer system 106 for further analysis.

In some implementations, the computer system 106 is configured to store a plurality of pre-determined X-ray diffraction patterns of known crystal structures or mineralogical composition of solids as a computer-readable database or a library. Each pre-determined X-ray diffraction pattern is corresponding to a type of solid object. The computer system 106 compares the stored database or the library with the generated X-ray diffraction pattern. Such comparison identifies the mineralogical composition and/or crystal structures present in the solids object, which determines a material of a solid object.

In some implementations, the computer system 106 includes a software that supports XRD pattern matching and have access to a database of XRD patterns. The computer system 106 can be configured to identify and extract peaks in the XRD patterns and then determine their positions and intensities. The computer system 106 can employ pattern matching algorithms to compare the identified peaks in the measured XRD pattern with those in the reference database. For example, pattern matching algorithms can include the Rietveld method, the Pawley method, or Le Bail refinement. During pattern matching, the computer system 106 fits the peaks of the measured XRD pattern to the reference XRD patterns in the database and evaluates the similarity between the measured and reference XRD patterns based on the goodness of fit. It can calculate a similarity score, which indicates how closely the patterns match. Higher similarity scores suggest better matches. The system can generate a list of potential matches from the database, ranked by their similarity to the measured XRD pattern. A mineralogical composition of the solid object can thus be determined using top ranking patterns.

In some implementations, in addition to qualitative identification, the computer system 106 can perform quantitative analysis to estimate the relative abundance of different crystal structures in the sample. This is achieved by comparing the intensities of diffraction peaks. For example, the computer system 106 can determine that a solid object includes 91.9% of dolomite [CaMg(CO₃)₂], 7.5% of calcite [CaCO₃] and 0.6% of quartz (SiO₂).

The digital imaging system 104 with a XRD system aids in determining the mineralogical composition of solid objects in the drilling fluids (e.g., the drill cuttings, additives, non-drilled solids). The digital imaging system 104 can be further integrated with a hyperspectral imaging technology, as described below, enabling the identification of both mineralogical and chemical/physical composition of the solids in drilling fluids. In some implementations, the computer system 106 has a real-time display of XRD patterns and/or compositions of solid materials. This enables mud engineers and drilling fluids specialists to promptly adjust additive concentrations or introduce new additives based on the composition analysis of solid objects, thereby enhancing wellbore stability. Such in-situ monitoring and analysis system allows for agile decision-making and adjustments during drilling operations.

In some implementations, the digital imaging system 104 includes a hyperspectral imaging system. Hyperspectral imaging is a technique that captures and processes information from across the electromagnetic spectrum, providing detailed spectral data for each pixel in an image. The system 100 including a hyperspectral imaging system can be configured to determine chemical and/or physical composition of the solid objects. The chemical composition can include a concentration of specific compounds or chemical elements in the solid objects. The physical composition can include properties such as texture, moisture content, porosity, density, crystallinity, or surface roughness.

In some implementations, the hyperspectral imaging system includes a light source, a dispersive element, and a detector. The light source is configured to produce an incidental light. The light source can be in the form of natural or artificial illumination, which is used to illuminate a target solid object. The illuminated solid object interacts with the incident light in a way that depends on its optical properties. This interaction includes absorption, reflection, transmission, and emission of light by the material of the solid object. The incident light turns into a response light after interaction with the solid object.

The dispersive element is configured to disperse the response light into a plurality of dispersed light with constituent wavelengths. Constituent wavelengths are the individual wavelengths that make up the dispersed light. When the dispersive element disperses the response light, it separates it into multiple wavelengths, and each of these individual wavelengths is considered a constituent wavelength. In an example, the dispersive element can be a prism or diffraction grating, which disperses the light into its various spectral components based on wavelength.

The dispersed light is subsequently focused onto a detector array, e.g., a CCD (Charge-Coupled Device) or CMOS (Complementary Metal-Oxide-Semiconductor) sensor. Each pixel in the detector array captures both spatial and spectral information. The detector is also configured to measure a light intensity of the dispersed light.

The resulting hyperspectral data can be visualized in the computer system 106 as a series of images, each representing the solid object at a specific wavelength or within a specific spectral range. In other words, hyperspectral data are information captured across a wide range of contiguous spectral bands or wavelengths. For example, hyperspectral imaging can capture bands spanning the ultraviolet, visible, and infrared regions of the electromagnetic spectrum. Each pixel in a hyperspectral image contains a spectrum of information, allowing for detailed analysis of the material composition of the imaged scene. The resulting hyperspectral image provides both spatial and spectral information for the solid object.

In some implementations, the hyperspectral images of solid objects are generated by the computer system 106 based on the light intensity of the dispersed lights transmitted from the digital imaging system 104. In some implementations, the hyperspectral images of solid objects are generated by the digital imaging system 104.

The computer system 106 can be configured to store reference spectra of known materials of solid objects as existing spectral libraries or databases. These libraries or database can include information about the characteristic absorption or reflection features of various compounds or chemical elements with various physical compositions (such as texture, moisture content, porosity, density, crystallinity, or surface roughness) across the electromagnetic spectrum. The computer system 106 can compare the acquired hyperspectral data with the reference spectra in these libraries to identify potential matches and determine the chemical and/or physical composition of the target solid object.

In some implementations, the computer system 106 utilizes spectral matching algorithms to compare the acquired spectra with known spectral signatures. In an example, the spectral matching algorithm includes a correlation analysis. Correlation analysis involves measuring the similarity between two spectra by calculating their correlation coefficient. A high correlation indicates a strong match, suggesting that the material in the sample can be similar to the reference material in the spectral library. In another example, the spectral matching algorithm includes a spectral unmixing, which is used when a pixel of a hyper hyperspectral contains a mixture of materials. The spectral unmixing can be utilized to estimate the abundance of each material in the pixel. It involves decomposing the mixed spectrum into a linear combination of member spectra. Each member spectrum represents a pure material, and their abundances are determined for each pixel. In some implementations, a machine learning approach is employed, such as supervised classification or deep learning. A model can be trained with labeled data (e.g., known spectra and corresponding materials) to learn the relationships between spectral features and material types. The trained model can then classify unknown spectra of solid objects and identify the materials of the solid objects.

In some implementations, the computer system 106 can be utilized to generate chemical maps that illustrate the spatial distribution of identified compounds within the solid objects. These maps can help visualize how different chemical components are distributed. In some implementations, the computer system 106 displays such maps in real-time on a screen.

The digital imaging system 104 with a hyperspectral imaging system assists in determining chemical and physical composition of solids in drilling fluids, facilitating an improved design of the additives added into the drilling fluid. Real-time display of this composition enables mud engineers and drilling fluids specialists to make timely adjustments, including modifying additive concentrations or introducing new additives. This adaptive approach contributes to enhancing wellbore stability throughout the drilling process.

In some implementations, the digital imaging system 104 includes a camera, an image sensor, vision sensor network or similar digital imaging device which can capture digital images of each solid object. The computer system 106 can receive the images, implement image processing techniques to determine the size, shape and/or quantity of the solids objects, and classify each image as either a drill cutting or a non-drilled solid. Such classification of solid objects has several applications, such as separate identification of cuttings and cavings for stuck pipe prevention, determination of cuttings concentration for effective hole cleaning assessment, identification of foreign objects in the wellbore such as metal shards or swarf associated with component failure, etc.

In some implementations, the computer system 106 can include algorithms to determine a long axis and a short axis for each solid object from the image of the solid object. Determining the long axis and the short axis for each of multiple images of solid objects enables the computer system 106 to determine a size for the solid objects carried past the digital imaging system 104. The computer system 106 can determine the size distribution and volume estimations additionally based on the likelihoods described earlier.

In some implementations, the computer system 106 can implement an artificial intelligence based model to analyze each image. In one example, the computer system 106 can implement a supervised learning model. For each type of solid object, the computer system 106 can use a set of images at the shale shaker containing cuttings to train a machine leaning (ML) or deep learning (DL) model. The ML or DL model is derived by using the set of photos from the expected cutting conditions to identify drill cuttings for a given drilling operation. For example, the computer system 106 can use regional convolutional neural networks (R-CNN) or other variations (for example, CNNs, faster R-CNNs) to automatically identify features describing the solid objects captured in the images. The computer system 106 can train the models to discriminate drill cuttings having expected cutting shapes and concentration from non-drilled solids. Additionally, the ML or DL models can be trained to classify (categorical or numerical) hole cleaning performance, cuttings concentration, among others.

In some implementations, the computer system 106 can count the number of solid objects in a window surrounding the digital images. The computer system 106 can compare the number of solid objects with a threshold solid object count, which is representative of a threshold dimension. That is, because dimensions of a drill cutting are expected to fall within a certain range, the computer system 106 can determine a threshold solid object count for an image of a drill cutting and store the threshold solid object count. The threshold solid object count can include an upper limit and a lower limit. Upon analyzing an image of a solid object received from the digital imaging system 104, the computer system 106 can determine the solid object count. If the solid object count falls within the upper limit and the lower limit of the threshold solid object count, the computer system 106 can associate an increased likelihood that the solid object in the image is a drill solid. Otherwise, the computer system 106 can determine that the solid object is a non-drilled solid.

In some implementations, the computer system 106 can further classify a type of the drilling solid into either drill cuttings or additives. As noted above, specific gravity and/or compositions of solids can be determined by the system, and each drilling cutting and additive has distinct specific gravity and compositions. Therefore, a further classification of drilling solids into either drilling cuttings or additives can rely on the specific gravity and/or compositions as a criteria.

In some implementations, the computer system 106 can further compare the estimated quantity of solid objects by the system with the cutting carrying index (CCI) to determine how good of the hole cleaning is. The CCI was developed empirically to provide a method of determining whether a hole was being cleaned efficiently. The equation for CCI is given below

$$CCI = ( K \times AV \times MW ) \div ( 400 , 000 ) \qquad ( 5 )$$

Where AV is annular velocity in ft/min, MW is mud weight in ppg, K is a Power Law Constant. The Power Law Constant K can be calculated using the equation (6) below.

$$K = 51 {1}^{1 - n} ( PV + YP ) \qquad ( 6 )$$

where PV is plastic viscosity in centipoise, YP is yield point in lb/100 sqft, and n is flow behavior index. The flow behavior index n can be determined by the equation (7) below

$$n = 3.3 \; 22 log \frac{( 2 PV + YP )}{( PV + YP )} \qquad ( 7 )$$

where PV is plastic viscosity in centipoise. YP is yield point in lb/100 sqft.

If the CCI index is equal to or less than 0.5, it indicates an inadequate hole cleaning efficiency. If the CCI index is equal to or greater than 1.0, it indicates a good hole cleaning efficiency. The multiple parameters used to calculate CCI (such as annular velocity, mud weight, plastic viscosity in centipoises) can be measured by an independent lab equipment different from the system 100. The quantitative assessment of cuttings by the system can then be cross-referenced with the CCI indication. For instance, if an independent laboratory equipment determines that the CCI index is over 1.0, it indicates an effective hole cleaning. In alignment with this, the system 100 should observe an anticipated volume of drill cuttings coming over the shale shaker. The anticipated volume can be based on drilling conditions like the rate of penetration. This real-time monitoring by the system 100 can thus be used to strengthen the reliability of the CCI calculation.

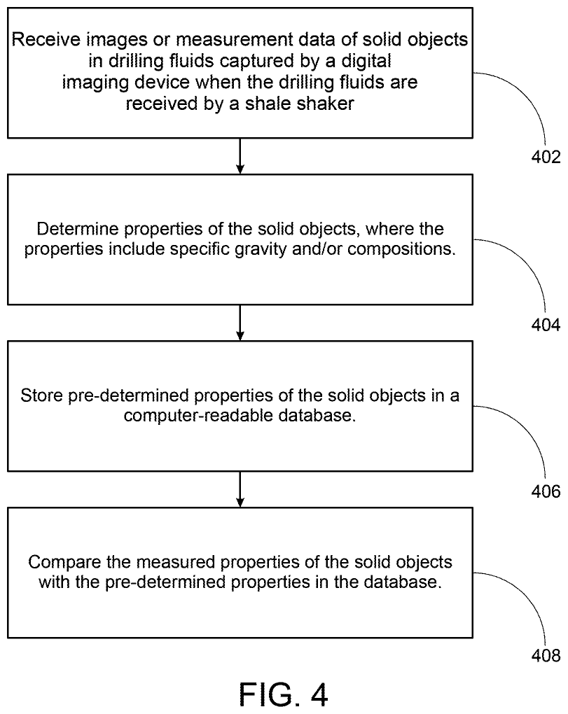

FIG. 4 is a flow chart of an example process for analysis of solid objects. At step 402, a computer system 106 receives images or measurement data of solid objects in drilling fluids captured by a digital imaging system 104. As shown in FIG. 1, the digital imaging system 104 is coupled to a shale shaker 102. The shale shaker 102 positioned at a surface of the Earth adjacent a wellbore and configured to receive a drilling fluid. The drilling fluids include a mixture of wellbore drilling mud and solid objects found in the wellbore while drilling the wellbore through a subterranean zone. The solid objects include drill solids and non-drilled solids. The drill solids include drill cuttings and additives. The digital imaging system 104 is configured to capture images or measurement data of the solid objects when the drilling fluid is received by the shale shaker 102.

At step 404, the computer system 106 determines one or more properties of solid objects. The properties include specific gravity and/or compositions. In addition, the properties can include size, shape, quantity, XRD patterns, or spectra data.

At step 406, the computer system 106 stores pre-determined properties of the solid objects as a computer-readable database or library.

At step 408, the computer system 106 compares the measured properties of solid objects with the stored properties in the database. The comparison can be used to determine a material composition and/or specific gravity of solid objects, a presence or absence of an additive, etc.

FIG. 5 illustrates a schematic diagram of an example computer system 106. The computer system 106 can include an interface 504 for network, a processor 506, an application layer 508, an additive data entry 510, a service layer 512, and a memory 502 for a network.

Memory 502, a computer-readable medium, stores and retrieves data for immediate use by the processor 506. It can include volatile and non-volatile memory 502. Volatile memory, such as RAM (Random Access Memory), provides quick access to data. Non-volatile memory 502 is a non-transitory computer-readable medium, like hard drives or SSDs, which retains data even when the power is off and is used for long-term storage. In some implementations, the non-volatile memory 502 stores the database of characteristics of the solid objects. The characteristics can include specific gravity, mineralogical compositions, chemical or physical compositions, XRD patterns, spectra, size, shape, threshold quantity. For example, as noted above, the memory 502 can store a table for specific gravity. The table can have a plurality of rows and columns where each row has at least a first column to represent a pre-determined specific gravity and a corresponding second column to represent an associated chemical or compounds for the solids. In one implementation, the non-volatile memory 502 stores measurement data or images transmitted from the digital imaging system 104. In another implementation, the memory 502 stores pre-determined system parameters for operation of X-ray or XRD systems, such as energy levels of X-ray beam, incident light beam intensity, etc.

The application layer 508 can include the software programs or applications installed on the computer. These applications enable users, e.g., mud engineers, to perform specific tasks, interact with data, and utilize various functionalities. The instructions or coding of software/applications can be stored in the non-volatile memory 502. In some implementations, the application layer 508 includes a pattern matching software program which is used to compare the measured XRD pattern with those in the reference database. As noted above, the pattern matching software can be configured to fit the peaks and compare them to reference XRD patterns. The software can further provide a list of potential matches ranked by their similarity to measured XRD pattern. In some implementations, the application layer 508 includes a spectral matching software to compare the acquired spectra by the hyperspectral imaging system with known spectral signatures, as discussed above. In an example, the spectral matching software includes a spectral unmixing algorithm, which is used when a pixel of a hyper hyperspectral contains a mixture of materials. The spectral unmixing can be utilized to estimate the abundance of each material in the pixel. It involves decomposing the mixed spectrum into a linear combination of member spectra. Each member spectrum represents a pure material, and their abundances are determined for each pixel.

The processor 506, i.e., the central processing unit (CPU), is responsible for executing instructions and managing the overall operation of the computer. It processes data received from the digital imaging system 104 and performs operations based on software instructions (e.g., the pattern matching software) that are stored in a computer-readable medium or memory 502. In an example, the processor 506 can calculate the specific gravity based on equation (1) and (2) discussed above and compare the calculated specific gravity to the table or database of the specific gravity stored in the memory 502 until it matches. From there, the processor 506 can access the memory 502 to obtain chemical or compounds for the solids in an associated column of the selected row.

The additive data entry 510 interface serves as a portal through which users, e.g., mud engineers, can input information. For example, the users can input operation parameters of digital imaging system 104, such as the X-ray energy levels, incident beam angle ranges, wavelength ranges, spectral or spatial resolutions for the hyperspectral images, camera angles and orientations, etc. In another example, the users can input parameters related to a drilling operations, such as drill bit details (for example, number of blades, type of bit, size, other drill bit details), drilling parameters (for example, rate of penetration, weight on bit, slurry flow-in/out, stand pipe pressure, other drilling parameters) and drilling mud details (for example, weight, oil-based or water-based, rheological properties, other drilling mud details) as inputs to the fully connected layer). These data can be used to generate anticipated characteristics of solid objects in the drilling fluids as they pass over the shale shaker. This includes projections regarding the anticipated size, shape, specific gravity, or compositions of the solids in the drilling fluids.

The interface 504 with the network enables the computer system 106 to connect with other devices, servers, or external networks, such as the digital imaging system 104. This connectivity allows for data exchange, updates, and remote access. For example, the computer system 106 can receive images or measurement data from the digital imaging system 104 wirelessly. The measurement data can include light intensity, thickness, spectra data or any other measurement data associated with the digital imaging system 104. The digital system can also transmit data (e.g., X-ray incident light intensity, X-ray energy levels, camera angles and orientations) to the digital imaging system 104 through the network interface 504. It is understood that the data exchange between the digital imaging system 104 and the computer system 106 can also be accomplished using cable wires.

The service layer 512 acts as a middleware that facilitates communication and interaction between different components of the computer system 106. It facilitates smooth data flow and functionality between the processor 506, application layer 508, and other system components. This layer can include various services or protocols that enable efficient data exchange and integration.

As noted above, the computer system 106 can receive images or measurement data captured by the digital imaging system 104 and determine properties of a target solid object. The properties include specific gravity and/or compositions. In addition, the properties can include quantity, size or shape of the solid objects. The computer system 106 can store pre-determined properties of the solid objects in a database or a library, such as specific gravity, compositions, XRD patterns or spectra, etc. The computer system 106 then compares one or more properties of the target solid object with the pre-determined properties in the database or the library. In some implementations, in response to a comparison result, the computer system 106 raises an alert to an operator that suggests a modification of a drilling parameter. The drilling parameter can be a concentration of an LCM. For example, the computer system 106 can assess if a particular LCM is stripped from the drilling fluid unexpectedly by determining the specific gravity and/or compositions of the solid objects in drilling fluids. If all anticipated LCMs are detected over the shale shakers, the computer system 106 can associate this detection with an increased likelihood that current LCM selection is appropriate and provides a recommendation to operators that it is unnecessary to add these LCMs in the subsequent well operations. On the other hand, if the absence of a particular LCM is detected, the computer system 106 can associate this detection with an increased likelihood that this particular LCM has obstructed the formations and raise an alert to operators indicating a potential need for adjusting the selection of LCMs or a concentration of an LCM in the subsequent well operation.

In some implementations, the computer system 106 includes a screen. The computer system 106 is configured to display the images or the measurement data captured by the digital imaging system 104 in real-time on the screen. This allows drill operators or mud engineers to promptly identify and address issues such as wellbore instability, unexpected formations, or equipment malfunctions.

### Implementations

Certain aspects of the subject matter described here can be implemented as a system for in-situ analysis of drilling fluid solids during wellbore drilling. The system includes a digital imaging system and a computer system. The digital imaging system is coupled to a shale shaker. The shale shaker is positioned at a surface of the Earth adjacent a wellbore and configured to receive a drilling fluid comprising a mixture of wellbore drilling mud and solid objects found in the wellbore while drilling the wellbore through a subterranean zone. The solid objects include additives. The digital imaging system is configured to capture at least one of images or measurement data of the solid objects when the drilling fluid is received by the shale shaker. The computer system is operatively coupled to the digital imaging system. The computer system includes one or more processors and a computer-readable medium storing instructions executable by the one or more processors to perform operations. The operations include receiving the at least one of images or measurement data captured by the digital imaging system and determining one or more properties of a target solid object of the solid objects. The one or more properties includes at least one of specific gravity or composition of the target solid object.

An aspect combinable with any other aspect includes the following features. The additives include a loss circulation material (LCM).

An aspect combinable with any other aspect includes the following features. The one or properties further include at least one of quantity, size, or shape.

An aspect combinable with any other aspect includes the following features. The digital imaging system includes an X-ray system configured to generate a first incident X-ray towards the target solid object of the solid objects, receive a transmitted X-ray that have passed through the target solid object and measure a light intensity of the transmitted X-ray.

An aspect combinable with any other aspect includes the following features. The computer system is configured to receive, from the digital imaging system, the light intensity of the transmitted X-Ray. The computer system is configured to determine the specific gravity of the target solid object using the equations:

$${μ}_{1} = - \frac{1}{t} ln ( \frac{I}{{I}_{0}} )$$

$$ρ = \frac{{μ}_{1}}{{μ}_{water}}$$

wherein ρ is specific gravity of the target solid object, I0 is light intensity of the first incident X-ray, I is light intensity of the transmitted X-ray, t is a thickness of the target solid object, μwater is linear attenuation coefficient of water, and μ1 is linear attenuation coefficient of the target solid object.

An aspect combinable with any other aspect includes the following features. The X-ray system is configured to generate a heterogeneous beam towards the target solid object. The heterogeneous beam includes the first incident X-ray at a first energy level E₁ and a second incident X-ray at a second energy level E₂. The computer system is configured to determine the specific gravity of the target solid object using the equations:

$$ρ = \frac{( {μ}_{1} - c {μ}_{2} )}{β ( 1 - c )}$$

$$where c = {( \frac{{E}_{2}}{{E}_{1}} )}^{3.1}$$

wherein ρ is the specific gravity of the target solid object, μ₁ is linear attenuation coefficient of the target solid object at the first energy level E₁, μ₂ is linear attenuation coefficient of the target solid object at the second energy level E₂, and β is scattering attenuation constant.

An aspect combinable with any other aspect includes the following features. The digital imaging system includes an X-ray Diffraction (XRD) system configured to emit a plurality of incident X-rays towards the target solid object of the solid objects at a plurality of incident angles. Each of the plurality of incident X-rays is corresponding to a respective incident angle of the plurality of incident angles. The XRD system is configured to receive a plurality of diffracted X-rays that have interacted with the target solid object and measure a light intensity for each of the plurality of diffracted X-rays.

An aspect combinable with any other aspect includes the following features. The computer system is configured to receive, from the digital imaging system, the measurement data of the light intensity of the plurality of diffracted X-rays. The computer system is configured to generate an X-ray diffraction pattern of the target solid object based on the light intensity of the plurality of diffracted X-rays and the plurality of incident angles. The computer system is configured to store a plurality of pre-determined X-ray diffraction patterns. Each of the plurality of pre-determined X-ray diffraction patterns is corresponding to a respective composition of the solid objects. The computer system is configured to compare the plurality of pre-determined X-ray diffraction patterns with the X-ray diffraction pattern of the target solid object to determine the composition of the target solid object.

An aspect combinable with any other aspect includes the following features. The digital imaging system includes a hyperspectral imaging system configured to produce an incidental light towards the target solid object of the solid objects. The hyperspectral imaging system is configured to disperse a response light that has interacted with the target solid object into dispersed lights with a plurality of constituent wavelengths, and measure a light intensity of the dispersed lights.

An aspect combinable with any other aspect includes the following features. The computer system is configured to receive, from the digital imaging system, the measurement data of the light intensity of the dispersed lights, and generate hyperspectral data of the target solid object based on the light intensity of the dispersed lights. The computer system is configured to store a plurality of pre-determined hyperspectral data. Each of the plurality of pre-determined hyperspectral data is corresponding to a respective composition of the solid objects. The computer system is configured to compare the plurality of pre-determined hyperspectral data with the hyperspectral data of the target solid object to determine the composition of the target solid object.

An aspect combinable with any other aspect includes the following features. The operations further include displaying the images or the measurement data captured by the digital imaging system in real-time.

An aspect combinable with any other aspect includes the following features. The operations further include: storing pre-determined properties of the solid objects, and comparing the one or more properties of the target solid object with the pre-determined properties.

An aspect combinable with any other aspect includes the following features. The operations further include raising an alert that recommends a modification of a drilling parameter based on a result of the comparison.

An aspect combinable with any other aspect includes the following features. The drilling parameter includes a concentration of a loss circulation material (LCM).

Certain aspects of the subject matter described here can be implemented as a method for in-situ analysis of drilling fluid solids during wellbore drilling. The method includes receiving, by one or more processors, at least one of images or measurement data captured by a digital imaging system coupled to a shale shaker and determining one or more properties of a target solid object of the solid objects. The one or more properties includes at least one of specific gravity or composition of the target solid object of the solid objects. The shale shaker is positioned at a surface of the Earth adjacent a wellbore. The shale shaker is configured to receive a drilling fluid comprising a mixture of wellbore drilling mud and solid objects found in the wellbore while drilling the wellbore through a subterranean zone. The digital imaging system is configured to capture at least one of images or measurement data of the solid objects when the drilling fluid is received by the shale shaker.

An aspect combinable with any other aspect includes the following features. The one or more properties further include at least one of quantity, size, or shape.

An aspect combinable with any other aspect includes the following features. The method includes classifying a type of the solid objects selected from a group including drill solids and non-drilled solids.

An aspect combinable with any other aspect includes the following features. The method includes storing pre-determined properties of the solid objects, and comparing the one or more properties of the target solid object of the solid objects with the pre-determined properties.

Certain aspects of the subject matter described here can be implemented as a non-transitory computer-readable medium storing instructions executable by one or more processors to perform operations. The operations include receiving, by one or more processors, at least one of images or measurement data captured by a digital imaging system coupled to a shale shaker of a wellbore drilling assembly. The shale shaker is positioned at a surface of the Earth adjacent a wellbore. The shale shaker is configured to receive a drilling fluid comprising a mixture of wellbore drilling mud and solid objects found in the wellbore while drilling the wellbore through a subterranean zone. The digital imaging system is configured to capture at least one of images or measurement data of the solid objects when the drilling fluid is received by the shale shaker. The operations include classifying a type of the solid objects selected from a group including drill solids and non-drilled solids, and determining one or more properties of a target solid object of the solid objects. The one or more properties includes at least one of specific gravity or composition of the target drill solid. The operations further include storing pre-determined properties of the solid objects and comparing the one or more properties of the target solid object with the pre-determined properties of the solid objects.

An aspect combinable with any other aspect includes the following features. The operations further include raising an alert that recommends a modification of a drilling parameter based on a result of the comparison.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing can be advantageous. Moreover, aspects described with reference to any figure or any implementation can be combined with aspects described with any other figure or any other implementation.

1. A system comprising:
a digital imaging system coupled to a shale shaker, the shale shaker positioned at a surface of the Earth adjacent a wellbore and configured to receive a drilling fluid comprising a mixture of wellbore drilling mud and solid objects found in the wellbore while drilling the wellbore through a subterranean zone, the solid objects comprising additives, the digital imaging system configured to capture at least one of images or measurement data of the solid objects when the drilling fluid is received by the shale shaker; and
a computer system operatively coupled to the digital imaging system, the computer system comprising one or more processors, and a computer-readable medium storing instructions executable by the one or more processors to perform operations comprising:
receiving the at least one of images or measurement data captured by the digital imaging system; and
determining one or more properties of a target solid object of the solid objects, the one or more properties comprising at least one of specific gravity or composition of the target solid object.

2. The system of claim 1, wherein the additives comprise a loss circulation material (LCM).

3. The system of claim 1, wherein the one or properties further comprise at least one of quantity, size, or shape.

4. The system of claim 1, wherein the digital imaging system comprises an X-ray system configured to generate a first incident X-ray towards the target solid object of the solid objects, receive a transmitted X-ray that have passed through the target solid object and measure a light intensity of the transmitted X-ray.

5. The system of claim 4, wherein the computer system is configured to receive, from the digital imaging system, the light intensity of the transmitted X-Ray, and determine the specific gravity of the target solid object using the equations:
$${μ}_{1} = - \frac{1}{t} ln ( \frac{I}{{I}_{0}} )$$
$$ρ = \frac{{μ}_{1}}{{μ}_{water}}$$
wherein ρ is specific gravity of the target solid object, I₀ is light intensity of the first incident X-ray, I is light intensity of the transmitted X-ray, t is a thickness of the target solid object, μ_{water} is linear attenuation coefficient of water, and μ₁ is linear attenuation coefficient of the target solid object.

6. The system of claim 5, wherein the X-ray system is configured to generate a heterogeneous beam towards the target solid object, the heterogeneous beam comprising the first incident X-ray at a first energy level E₁ and a second incident X-ray at a second energy level E2, and wherein the computer system is configured to determine the specific gravity of the target solid object using the equations:
$$ρ = \frac{( {μ}_{1} - c {μ}_{2} )}{β ( 1 - c )}$$
$$where c = {( \frac{{E}_{2}}{{E}_{1}} )}^{3.1}$$
wherein ρ is the specific gravity of the target solid object, μ₁ is linear attenuation coefficient of the target solid object at the first energy level E₁, μ₂ is linear attenuation coefficient of the target solid object at the second energy level E₂, and β is scattering attenuation constant.

7. The system of claim 1, wherein the digital imaging system comprises an X-ray Diffraction (XRD) system configured to emit a plurality of incident X-rays towards the target solid object of the solid objects at a plurality of incident angles, each of the plurality of incident X-rays corresponding to a respective incident angle of the plurality of incident angles, receive a plurality of diffracted X-rays that have interacted with the target solid object and measure a light intensity for each of the plurality of diffracted X-rays.

8. The system of claim 7, wherein the computer system is configured to receive, from the digital imaging system, the light intensity of the plurality of diffracted X-rays, and generate an X-ray diffraction pattern of the target solid object based on the light intensity of the plurality of diffracted X-rays and the plurality of incident angles, and
wherein the computer system is configured to store a plurality of pre-determined X-ray diffraction patterns, each of the plurality of pre-determined X-ray diffraction patterns corresponding to a respective composition of the solid objects, and compare the plurality of pre-determined X-ray diffraction patterns with the X-ray diffraction pattern of the target solid object to determine the composition of the target solid object.

9. The system of claim 1, wherein the digital imaging system comprises a hyperspectral imaging system configured to produce an incidental light towards the target solid object of the solid objects, disperse a response light that has interacted with the target solid object into dispersed lights with a plurality of constituent wavelengths, and measure a light intensity of the dispersed lights.

10. The system of claim 9, wherein the computer system is configured to receive, from the digital imaging system, the light intensity of the dispersed lights, and generate hyperspectral data of the target solid object based on the light intensity of the dispersed lights, and
wherein the computer system is configured to store a plurality of pre-determined hyperspectral data, each of the plurality of pre-determined hyperspectral data corresponding to a respective composition of the solid objects, and compare the plurality of pre-determined hyperspectral data with the hyperspectral data of the target solid object to determine the composition of the target solid object.

11. The system of claim 1, wherein the operations further comprise displaying the images or the measurement data captured by the digital imaging system in real-time.

12. The system of claim 1, wherein the operations further comprise: storing pre-determined properties of the solid objects, and comparing the one or more properties of the target solid object with the pre-determined properties.

13. The system of claim 12, wherein the operations further comprise raising an alert that recommends a modification of a drilling parameter based on a result of the comparison.

14. The system of claim 13, wherein the drilling parameter comprises a concentration of a loss circulation material (LCM).

15. A method, comprising:
receiving, by one or more processors, at least one of images or measurement data captured by a digital imaging system coupled to a shale shaker, the shale shaker positioned at a surface of the Earth adjacent a wellbore and configured to receive a drilling fluid comprising a mixture of wellbore drilling mud and solid objects found in the wellbore while drilling the wellbore through a subterranean zone, the digital imaging system configured to capture at least one of images or measurement data of the solid objects when the drilling fluid is received by the shale shaker; and
determining one or more properties of a target solid object of the solid objects, the one or more properties comprising at least one of specific gravity or composition of the target solid object of the solid objects.

16. The method of claim 15, wherein the one or more properties further comprise at least one of quantity, size, or shape.

17. The method of claim 15, further comprising classifying a type of the solid objects selected from a group including drill solids and non-drilled solids.

18. The method of claim 15, further comprising storing pre-determined properties of the solid objects, and comparing the one or more properties of the target solid object of the solid objects with the pre-determined properties.

19. A non-transitory computer-readable medium storing instructions executable by one or more processors to perform operations comprising:
receiving, by one or more processors, at least one of images or measurement data captured by a digital imaging system coupled to a shale shaker of a wellbore drilling assembly, the shale shaker positioned at a surface of the Earth adjacent a wellbore and configured to receive a drilling fluid comprising a mixture of wellbore drilling mud and solid objects found in the wellbore while drilling the wellbore through a subterranean zone, the digital imaging system configured to capture at least one of images or measurement data of the solid objects when the drilling fluid is received by the shale shaker;
classifying a type of the solid objects selected from a group including drill solids and non-drilled solids;
determining one or more properties of a target solid object of the solid objects, the one or more properties comprising at least one of specific gravity or composition of the target drill solid;
storing pre-determined properties of the solid objects; and
comparing the one or more properties of the target solid object with the pre-determined properties of the solid objects.

20. The medium of claim 19, wherein the operations further comprise raising an alert that recommends a modification of a drilling parameter based on a result of the comparison.
